(19) United States
(12) Patent Application Publication
(10) Pub. No.: US 2025/0299822 A1
(43) Pub. Date: Sep. 25, 2025

(54) DIAGNOSING CONCOMITANT FIBROMYALGIA AND LUPUS AND DETERMINING TREATMENT EFFICACY

(71) Applicant: Robert Katz, Chicago, IL (US)

(72) Inventor: Robert Katz, Chicago, IL (US)

(21) Appl. No.: 19/223,235

(22) Filed: May 30, 2025

Related U.S. Application Data
(63) Continuation of application No. PCT/US2023/082522, filed on Dec. 5, 2023.
(60) Provisional application No. 63/387,808, filed on Dec. 16, 2022.

(51) Int. Cl. G16H 50/20; G16H 10/20; G16H 20/10

(52) U.S. Cl. CPC G16H 50/20; G16H 10/20; G16H 20/10

(57) ABSTRACT

The present invention comprises excluding fibromyalgia patients in clinical studies for patients with lupus, treating the patients for lupus, and increasing the effectiveness of the lupus treatment by excluding patients with fibromyalgia. This makes the SELENA-SLEDAI and BILAG unreliable instruments for assessing the effectiveness of lupus interventions.

### BACKGROUND

The present application claims priority to PCT Application Ser. No. PCT/US2023/082522, filed Dec. 5, 2023, which claims priority to U.S. provisional application Ser. No. 63/387,808, filed Dec. 16, 2022, each herein incorporated by reference in their entireties.

### BACKGROUND

The invention generally relates to methods and systems for diagnosing and treating lupus and fibromyalgia. PCT Application Number: PCT/US2023/082522

The high failure rate in clinical drug trials for systemic lupus erythematosus (SLE) is perplexing. Belimumab is the only therapeutic agent in the last six decades to be approved for the treatment of systemic lupus erythematosus (Mahieu, et al., 2016). The failure of patients with lupus to beneficially respond to new medications has largely been attributed to the complexity of lupus and to methodological shortcomings.

A proposed limitation of SLE drug clinical trials is they are not targeting appropriate clinical samples. A factor not appreciated in most lupus drug failures is the relevance of a hidden pathology operating in the clinical trial: fibromyalgia syndrome (FMS).

Part of the reason that some lupus trials are inconclusive is because fibromyalgia patients are not excluded from the assessment. In an extensive evaluation of lupus patients (Wolfe, 2014) given the diagnosis of lupus by a rheumatologist, 31 percent also met the criteria for fibromyalgia based on the 2010 ACR guidelines for the diagnosis. An understanding of how fibromyalgia syndrome symptoms can react to drug treatments of lupus can give researchers the ability to develop therapies that can isolate lupus or treat the symptoms of both diseases.

Diagnosing concomitant fibromyalgia in lupus patients poses problems in clinical trials because lupus and concomitant fibromyalgia share many symptoms. They bear a close resemblance with respect to pain, fatigue, stiffness, headaches, swelling and edema, fever, mouth sores, and hair loss. Because of the overlapping nature of these symptoms, changes related to concomitant fibromyalgia may contribute to a misrepresentation of lupus disease activity. For example, in a major fibromyalgia flare, elevations in reported symptoms may masquerade as lupus flares.

Importantly, the symptoms of fibromyalgia are not likely to respond to lupus therapy and if unnoticed will spoil the success of a drug trial.

Another prominent feature of concomitant fibromyalgia when coupled with another rheumatic disease is disease amplification. In several studies, the presence of concomitant fibromyalgia with another rheumatic disease adds a high level of measured disease activity to manifestations of the clinical disorder. This is an example of disease amplification, a complication often associated with less favorable outcomes in medical research (Chen, et al., 2016) (Wolfe, Fibromyalgia/SLE, 2009).

Lupus samples in clinical trials are problematic when many of the patients in the sample contain a critical containment of concomitant fibromyalgia that is not targeted by new therapeutic agents. Because of the mix of the two pathologies operating, treatment-related changes and variables used to track improvement may not accurately measure changes in the targeted pathology. The pharmaceutical agent could beneficially affect the targeted pathology but provoke an adverse reaction in the non-targeted pathology, the net result being that the therapeutic benefits to the targeted pathology (SLE) are wiped out by off-targeted effects from fibromyalgia.

At minimum, failure to take concomitant fibromyalgia into account is likely to result in measured outcome variables that are crucial for establishing the value of new therapeutic agents. Failure to make the distinction between SLE and SLE coupled with concomitant fibromyalgia can create measurement errors that covertly mask real benefits of the clinical drug trial. An important task in future efficacy research with SLE will be to screen out those with concomitant fibromyalgia before the clinical trial starts.

The present invention attempts to shed light on the clinical impact of concomitant fibromyalgia based on the efficacy of standard care medication in a real-life setting and to solve these problems as well as others.

### SUMMARY OF THE INVENTION

Provided herein are systems for methods for diagnosing concomitant fibromyalgia and determining the masked therapeutic benefits of medication in response to lupus therapy or treatment; and determining the standard of care medication by less favorable clinical outcomes when systemic lupus is coupled with concomitant fibromyalgia.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a graph of the SELENA SLEDAI Symptoms in FMS Patients.

FIG. 2 is a graph of the SLEDAI symptoms in Fibromyalgia patients.

FIG. 3 is a graph showing the pain increases in severity as PDS scores increase.

FIG. 4 is a graph showing the response to treatment decreases as PDS scores increase.

FIG. 5 is a graph showing the DAS score in rheumatoid arthritis patients was 2.82 (0.49 to 6.93), and the DAS score mean was 3.45 and varied from 1.25 to 4.63.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### INTRODUCTION

Fibromyalgia (FMS) is a syndrome characterized by chronic musculoskeletal pain and a range of associated symptoms, including muscle and joint stiffness, insomnia, fatigue, and cognitive dysfunction. (1) It was first described in the 19th century, and its central nervous system involvement was recognized in the 1970s and 1980s. (2) In 1990, the American College of Rheumatology (ACR) established diagnostic criteria, which have been updated recently. (3) These criteria include bilateral pain above and below the waist and chronic generalized pain lasting at least three months, with pain on palpation in at least 11 of 18 specific body sites. (4) FM affects approximately 5% of the global population, with a higher incidence in women and typically appearing between the ages of 30 and 35. (5) Despite its prevalence, FM remains a poorly understood and challenging condition to diagnose and treat.

Because of the poor understanding of this condition, FMS often goes unnoticed when combined with other rheumatic diseases. Other diseases found to display comorbidity with fibromyalgia include systemic lupus erythematosus (SLE), psoriatic arthritis, rheumatoid arthritis, and ankylosing spondylitis. (6, 7) The prevalence of FMS in patients diagnosed with SLE specifically has been the focus of numerous studies. Middleton et al. concluded that 22% of patients with SLE met the American College of Rheumatology's (ACR) criteria for FMS, and another 23% of patients with SLE were diagnosed with FMS but did not meet the clinical criteria. (8) Thus, for up to 45% of patients diagnosed with SLE, there is a second pathology in operation that goes unnoticed in experimental designs. In other studies, Gladman et al. (9) noted that 22% of 119 clinic patients with SLE also had symptoms of FMA; the John Hopkins Lupus Cohort (10) found that 17.3% of 173 patients with SLE had 11 or more tender points, which is diagnostic of FMS; Handa et al. (11) reported FMS symptoms in 8.2% of 158 patients with SLE; and Valencia-Flores et al. (12) found that 9.5% of 106 patients with SLE also satisfied the ACR criteria for FMS.

Another prominent feature of concomitant FMS when coupled with other rheumatic diseases is disease amplification, which is often associated with less favorable outcomes in medical research. In several studies, the presence of concomitant FMS with another rheumatic disease adds a high level of measured disease activity to the manifestations of the clinical disorder. (13) For example, a two-fold increase in pain, fatigue, and disability was encountered in those lupus and concomitant FMS patients, suggesting a potential for misleading symptoms to affect the clinical picture of lupus.

Patients with FMS frequently report symptoms that are part of the SELENA-SLEDAI and BILAG disease activity scales used to measure SLE severity. (14) Lupus patients with concomitant FMS may report these symptoms more commonly, and they may appear to be resistant to lupus treatment. This has an important impact on clinical drug trials because the inclusion of patients with SLE and FMS together can lead to an apparent lack of efficacy for new drugs.

### Definitions

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the terms “therapies” and “therapy” can refer to any method(s), composition(s), and/or agent(s) that can be used in the prevention, treatment and/or management of a disease or condition, such as neoplasia or infection, or one or more symptoms thereof.

As used herein, the terms “treat,” “treatment,” and “treating” in the context of the administration of a therapy to a subject refer to the reduction or inhibition of the progression and/or duration of a disease or condition, such as neoplasia or infection, the reduction or amelioration of the severity of a disease or condition, such as neoplasia or infection, and/or the amelioration of one or more symptoms thereof resulting from the administration of one or more therapies. Such treatment regimens may include, but are not limited to, immunosuppressants (ex: cyclophosphamide, corticosteroids, mycophenolate, etc.) and/or disease modifying antirheumatic drugs (DMARDs; ex: methotrexate, azathioprine, leflunomide, belimumab, and antimalarials such as PLAQUENIL® and hydroxychloroquine).

As used herein, “diagnosis” or “diagnostic” means a prediction of the type of disease or condition from a set of marker values and/or patient symptoms.

### DESCRIPTION OF EMBODIMENTS

Symptoms of fibromyalgia can sometimes resemble those of other rheumatic diseases including lupus. Because many patients with lupus have concomitant fibromyalgia, it is difficult to separate the fibromyalgia component. Fibromyalgia symptoms can easily contaminate lupus activity indices including the SELENA-SLEDAI and the BILAG. The SELENA-SLEDAI is a tool for measuring the activity of systemic lupus and is frequently used in clinical drug trials. BILAG (British Isles Lupus Assessment Group) is a validated global measure of treatment response in systemic lupus erythematosus (SLE) clinical trials but does not include patient-reported outcomes. The present invention comprises modifying indices in SELENA-SLEDAI and BILAG in clinical studies that include lupus patients and those with concomitant fibromyalgia; and further refining assessment tools in response to a treatment for lupus.

The present invention further comprises excluding patients with concomitant fibromyalgia in clinical studies that include lupus patients, and making the SELENA-SLEDAI and the BILAG more accurate assessment tools and exclude patients who are not as likely to respond to new lupus treatment.

The present invention comprises using the 2010 and 2016 American College of Rheumatology criteria for the diagnosis of fibromyalgia in clinical studies for lupus patients, wherein the 2010 and 2016 American College of Rheumatology (ACR) criteria for the diagnosis of fibromyalgia includes a one-page form for assessing the presence of the illness, concomitant fibromyalgia to be determined in lupus patients. The present invention further comprises using the ACR diagnostic criteria scale and those patients who scored 12 or more points on the ACR diagnostic criteria scale are diagnosed with having fibromyalgia. In one embodiment, the ACR diagnostic criteria scale include patients who scored 10 to 12 on that scale and are considered to be intermediate in the determination of a fibromyalgia diagnosis. In one embodiment, the present invention further comprises evaluating patients with inflammatory rheumatic diseases in addition to fibromyalgia (concomitant fibromyalgia) for treatment response based on a visual analog scale.

The present invention comprises excluding fibromyalgia patients in clinical studies for patients with lupus, treating the patients for lupus, and increasing the effectiveness of the lupus treatment by excluding patients with fibromyalgia. This makes the SELENA-SLEDAI and BILAG unreliable instruments for assessing the effectiveness of lupus interventions.

The examples below highlight the significant impact of concomitant FMS on patients with rheumatic diseases. Among the patients in the examples, those diagnosed with FMS, as well as those with other rheumatic diseases and concomitant FMS, reported higher pain levels and worse response to treatment compared to non-FMS rheumatic disease patients without concomitant FMS. These findings underscore the substantial burden of pain and lack of treatment response experienced by individuals with concomitant FMS. It is important to recognize and address FMS in patients with other rheumatic diseases to tailor more effective pain management strategies and improve treatment outcomes, as well as be aware of its presence in future pharmaceutical trials.

### Overview of SELENA-SLEDAI

The SLEDAI, established in 1985, is a global assessment tool designed for evaluating lupus disease activity over the past ten days. (22) Comprising 24 clinical and laboratory variables spanning nine organ systems, this index was developed through expert consensus in rheumatology. Weightings for each parameter were determined using regression models, with input from clinicians' global judgment. Descriptor scores vary from 1 to 8, and the maximum attainable score for all 24 descriptors is 105.

One noteworthy aspect of SLEDAI is its incorporation of clinical judgment. This means that a clinician's expertise and evaluation of the patient's condition play a role in the assessment. While the index provides valuable objective data, it also acknowledges the importance of clinical judgment, which can take into account subtleties and nuances that may not be fully captured by laboratory results alone.

A revised edition of the SLEDAI, known as SELENA-SLEDAI, was developed specifically for the Safety of Estrogens in Lupus National Assessment (SELENA) study. This updated version introduced several enhancements, including the incorporation of a glossary and adjustments to the scoring system to address the persistence of active disease in certain descriptors (such as rash, mucosal ulcers, and alopecia). Previously, these descriptors were only scored if they were either new or recurring. Within the SELENA-SLEDAI framework, researchers adopted a more inclusive approach when evaluating descriptor presence. They considered the presence of either objective or subjective findings as sufficient criteria for scoring a descriptor as being present. (23) This modification aimed to provide a more comprehensive and nuanced assessment of lupus disease activity in the context of the SELENA study.

In 2002, the SLEDAI-2000, often referred to as SLEDAI-2K, was introduced as a tool for assessing global disease activity. (24) SLEDAI-2K represents a modification of the original SLEDAI, designed to allow for the documentation of ongoing disease activity in certain descriptors, including rash, alopecia, mucosal ulcers, and proteinuria. This adaptation was made to provide a more comprehensive evaluation of disease activity. Extensive validation studies have been conducted to compare SLEDAI-2K with the classic SLEDAI, demonstrating that SLEDAI-2K is sensitive to changes in disease activity over time. Furthermore, SLEDAI-2K has proven to be a robust predictor of mortality in individuals with SLE.

All SLEDAI versions have undergone validation and are widely employed by researchers in both clinical and research settings. SLEDAI's practicality in clinical application, case of administration, and straightforward scoring system make it a cornerstone in lupus assessment. Notably, SLEDAI-2K stands out as one of the most frequently utilized tools for evaluating global disease activity in longitudinal observational studies and clinical trials.

However, the SLEDAI versions have limitations. They do not capture improvements or deteriorations in a patient's condition, lack granularity in assessing severity within specific organ systems, and demonstrate reduced sensitivity to change when compared to alternative instruments. Additionally, relying solely on SLEDAI as a determinant of disease flare or worsening has its constraints. This is because the SLEDAI score remains unchanged in cases of worsening preexisting symptoms or when symptoms have improved but not completely resolved. To address this limitation, the SELENA-SLEDAI introduced a distinct flare index to better capture and assess disease flare in lupus patients.

Another limitation of the SLEDAI index is that it does not appear to account for fatigue in SLE when measuring disease activity. (25) Fatigue is one of the most frequently reported symptoms of patients with FMS, and therefore, using a scale that fails to consider this item can result in the misclassification of patients with both SLE and FMS.

This example addresses the high failure rate observed in clinical drug trials for Systemic Lupus Erythematosus (SLE) and investigates the underappreciated influence of concomitant fibromyalgia syndrome (FMS) in these trials. With only a limited number of drugs approved for SLE treatment in the past six decades, the complexity of SLE and the challenges in measuring disease activity have been identified as contributing factors to these failures. There exists a coexistence of FMS with SLE that often leads to overlapping symptoms such as pain and fatigue, which can confound the assessment of disease activity using established tools like the SELENA-SLEDAI index. Furthermore, a significant proportion of SLE patients may have undiagnosed FMS, which can affect the interpretation of clinical trial results. Results of an in-office study examining the presence of SELENA-SLEDAI symptoms in patients diagnosed with FMS are presented and highlight the overlap of some SLE and FMS symptoms. An example of a failed lupus drug trial is included to demonstrate how concomitant FMS may have contributed to trial failures by masking the therapeutic benefits of medications. Enhancing the precision of disease activity measurement and optimizing the effectiveness of SLE treatments calls for the development of well-defined assessment tools, including a straightforward questionnaire for fibromyalgia diagnosis, and trial designs that carefully consider the coexistence of FMS.

### Overview of BILAG

The development of an ideal systemic lupus erythematosus (SLE) disease activity index (DAI) involved several stages. Between the 1950s and 1980s, approximately 60 different DAIs were created, but none were reliable or validated. (21) Successful attempts to define disease activity began in the 1980s.

In Toronto, Murray Urowitz, Dafna Gladman, and their colleagues introduced the SLE Disease Activity Index (SLEDAI), which proved to be reliable, valid, and sensitive to changes in disease activity. (22) In Boston, Liang et al. (21) developed the Systemic Lupus Activity Measure (SLAM), Bombardieri proposed the European Consensus Lupus Activity Measurement, and Smolen introduced the National Institutes of Health SLE Index Score. (23)

In the UK in 1984, the BILAG (British Isles Lupus Assessment Group) recognized that a global score was inadequate to capture the full range of real-life lupus activity. They aimed to create an index that could track changes in disease activity over time and differentiate between partial or complete improvement and deterioration. This new approach was based on the physician's intention to treat, acknowledging that different patients might require different treatment strategies based on their unique disease manifestations and progression. (24)

The BILAG index focuses on assessing disease activity in eight specific organs or systems: constitutional, mucocutaneous, neurological, musculoskeletal, cardiovascular/respiratory, vasculitis, renal, and hematological. There are two versions of the BILAG index: the classic BILAG index and the revised BILAG-2004 index.

The classic BILAG index is a categorical scale with up to 18 questions in each category. (25, 26) It can be quickly completed in less than 5 minutes on paper or a computer, with urine and blood test results added later. Accurate training is crucial, and the key to a precise BILAG assessment is scoring a clinical feature only if the physician is certain it is due to lupus activity. For each symptom or sign attributed to lupus activity, it is determined whether the feature is the same, better, worse, or new over the past 4 weeks compared to the previous 4 weeks. These recorded items are then converted into A-E scores for each organ/system.

Scoring for the BILAG Disease Activity Index (DAI) is based on the concept of “intention to treat.” An A (action) score indicates that the patient requires treatment with at least prednisolone 20 mg daily or immunosuppressive therapy. A B (beware) grade is assigned to patients requiring a lower level of immunosuppression. A C grade implies contentment, indicating low disease activity requiring minimal treatment. A D (discount) grade means that the patient had been active in the past but is no longer active, and an E (never active) grade suggests that the disease has never been active in that particular organ/system.

Issues with the classic BILAG index became apparent over time. One problem was that if there was an improvement in an item that had previously scored grade A (indicating the need for treatment), it would be downgraded to a C on the next assessment four weeks later. If the item remained the same, it would score a B on the third assessment, reflecting a poor response to treatment. This pattern was consistent with the intention-to-treat premise on which the BILAG index is based, suggesting that more treatment is required for further improvement. However, this could give a false impression of deterioration due to the increase in the score.

Additionally, in order to assess a disease flare, it was necessary to identify items that were recorded as new or worse. Some items in the index were capturing damage (e.g., avascular necrosis) rather than disease activity. In the renal assessment, it did not account for stable proteinuria, only worsening proteinuria, and it was not validated using methods other than 24-hour protein values, such as the urine protein-to-creatinine ratio. The classic BILAG index also did not adequately capture many gastrointestinal and ophthalmic manifestations, had outdated neuropsychiatric terminology, and lacked an adequate glossary for consistent interpretation.

The classic BILAG index underwent a revision and was replaced by the BILAG-2004 index, which brought several improvements: (27)

The BILAG-2004 index categorized nine organs/systems instead of eight, with the addition of ophthalmic and gastrointestinal systems. The vasculitis section was removed, and its items were integrated into the relevant systems. The mucocutaneous and neurological systems were organized more logically, and renal disease was better assessed.

Features indicating damage, such as sclerodactyly, avascular necrosis, calcinosis, and tendon contractures, were removed from the index. Fatigue and migraine were also excluded.

The glossary accompanying the BILAG-2004 index was more standardized and comprehensive. It featured updated terminology and clearer definitions of various terms.

The scoring system was improved in the BILAG-2004 index. Items previously scoring A could now improve to score B instead of C. Grade A was defined for patients requiring treatment with medium/high doses of steroids or immunosuppressive drugs and/or high-dose anticoagulation (international normalized ratio >3). This modification aimed to better reflect changes in disease activity and response to treatment.

The discrepancy between real-world clinical practice (for example, patients have shown positive responses to rituximab treatment) and the outcomes of clinical trials is a persistent issue. (28) Many studies using the classic BILAG index have demonstrated rituximab's effectiveness in treating patients with refractory SLE. (29-32) However, the failure of rituximab in clinical trials appears to have multiple contributing factors. (33) In trials like the EXPLORER and LUNAR trials, patients were often on high doses of corticosteroids and immunosuppressive drugs, which could have influenced the trial outcomes. Nonetheless, there may also be other issues related to clinical trial design that need to be considered.

This invention addresses the high failure rate observed in clinical drug trials for Systemic Lupus Erythematosus (SLE) and investigates the underappreciated influence of concomitant fibromyalgia syndrome (FMS) in these trials. With only a limited number of drugs approved for SLE treatment in the past six decades, the complexity of SLE and the challenges in measuring disease activity have been identified as contributing factors to these failures. The study highlights that the coexistence of FMS with SLE often leads to overlapping symptoms such as pain and fatigue, which can confound the assessment of disease activity using established tools like the BILAG index. Furthermore, it reveals that a significant proportion of SLE patients may have undiagnosed FMS, which can significantly affect the interpretation of clinical trial results. Results of an in-office study examining the presence of BILAG symptoms in patients diagnosed with FMS are presented and highlight the overlap of some SLE and FMS symptoms. Examples of previous drug trials, such as abatacept and epratuzumab, are presented to demonstrate how concomitant FMS may have contributed to trial failures by masking the therapeutic benefits of medications. The study emphasizes the need for more sophisticated assessment tools and trial designs that account for the presence of concomitant FMS to improve the accuracy of disease activity measurement and enhance the effectiveness of SLE treatments.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: The Effect of Concomitant Fibromyalgia on SELENA-SLEDAI Assessment of Lupus Activity

### In-Office Study of SELENA-SLEDAI Questionnaire

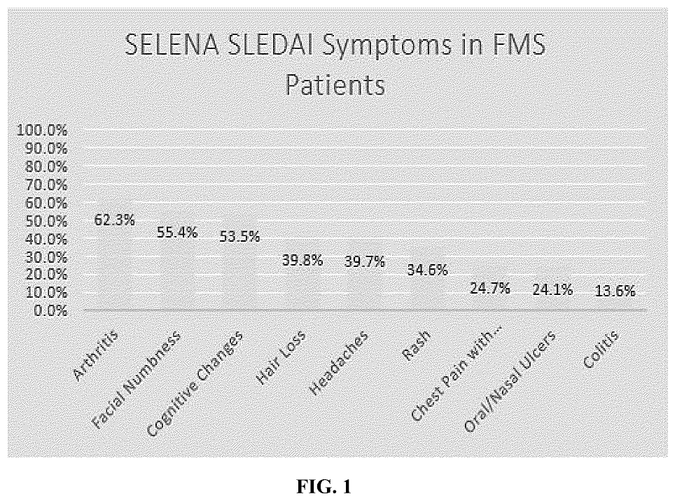

A recent study was conducted to assess the value of the SELENA-SLEDAI index in patients with lupus and concomitant fibromyalgia. (26) 191 patients with FMS took an in-office questionnaire asking whether they experienced lupus-like symptoms that are listed on the SELENA-SLEDAI index. 119 patients (62% of the sample) reported experiencing “arthritis”-like symptoms, 106 (55%) reported facial numbness, 102 (54%) reported cognitive changes, 76 (40%) reported hair loss, 74 (40%) reported severe persistent headaches, 66 (35%) reported a rash, 47 (25%) reported chest pain with deep breathing, 46 (24%) reported oral or nasal ulcers, and 26 (14%) reported colitis. These results are illustrated in FIG. 1.

### Methods

The fibromyalgia and non-fibromyalgia syndrome rheumatic disease patient groups did not significantly differ in age or education. There were 191 patient (160 females and 31 males) and 130 patients (82 females and 48 males) in the fibromyalgia and non-fibromyalgia groups respectively. The mean age for the fibromyalgia patients was 52.45 years for females and 50.39 years for males. The mean age for the control group was 55.23 years for females and 54.02 years in males. The mean pain level using a visual analog scale (VAS) of 0 to 10 was 6.45 in fibromyalgia and 4.6 in controls. Concentration difficulty was 6.0 in fibromyalgia and 4.3 in controls. Fatigue was 6.2 in fibromyalgia and 4.9 in controls. Sleep disruption was 5.9 in fibromyalgia and 4.8 in controls.

SELENA-SLEDAI and BILAG (British Isles Lupus Assessment Group) scores were measured in patients with lupus and concomitant fibromyalgia. Patients were given an in-office questionnaire in which the SELENA-SLEDAI symptoms and the BILAG symptoms were listed.

As shown in FIG. 1, analyzing the fibromyalgia syndrome sample, the SELENA-SLEDAI results were 119 arthritis patients (62.3 percent); 102 patients with cognitive changes (53.5 percent); 74 patients with severe persistent headaches (39.7 percent); 76 patients with hair loss (39.8 percent); 46 patients with oral or nasal ulcers (24.1 percent); 106 patients with facial numbness (55.4 percent); 47 patients with chest pain with deep breathing (24.7 percent); 26 patients with colitis (13.6 percent); and 66 patients with rash (34.6 percent).

Forty-three patients with FMS (fibromyalgia syndrome) were administered the BILAG (British Isles Lupus Assessment Group) by a rheumatologist experienced in the use of this study instrument. None of these patients had lupus.

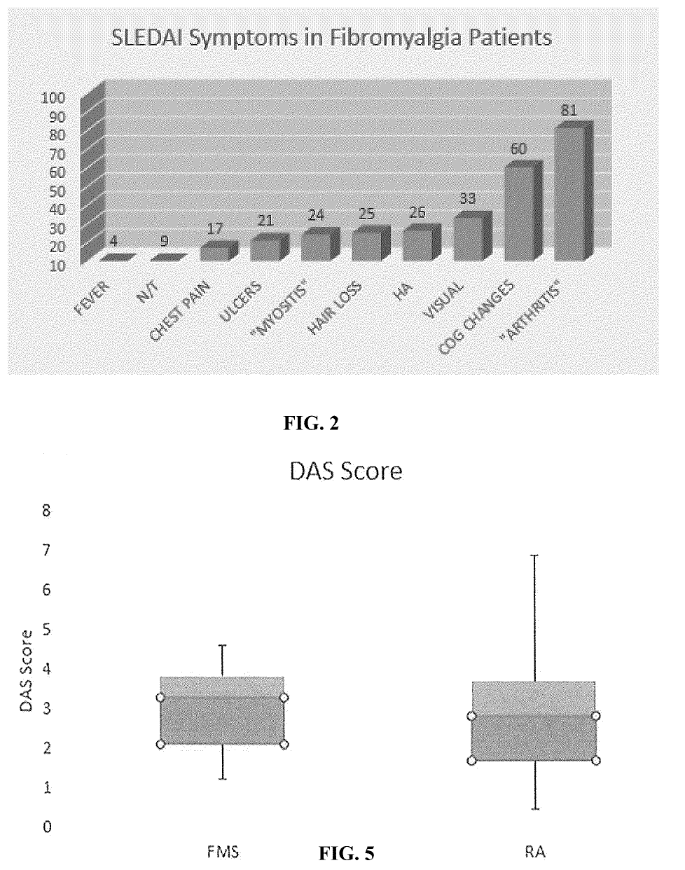

Several fibromyalgia syndrome patients were also evaluated using the SELENA-SLEDAI disease activity index, the SLAM (Systemic Lupus Activity Measure), and the LAI (Lupus Activity Index). FIG. 2 is a graph of the SLEDAI symptoms in Fibromyalgia patients.

### Results

Symptoms experienced by 43 fibromyalgia patients in response to questions from the BILAG included arthralgias (40 patients); myalgias (40 patients); fatigue (34 patients); episodic migrainous headaches (33 patients); small mucosal ulcers (16 patients); dyspnea (18 patients); nausea (18 patients); intermittent chest pain (16 patients); Raynaud's (7 patients); alopecia (5 patients); tendinitis (4 patients); rash (9 patients); swollen fingers (10 patients); paresthesias (9 patients); lymphadenopathy (7 patients); and weight loss (2 patients). Patients sometimes endorsed other symptoms, but not commonly.

The results among 20 patients who were evaluated with the SLEDAI were altered memory, inability to sustain attention, and possible organic brain syndrome (8 patients); lupus headaches (9 patients); mucosal ulcers (8 patients); rash (6 patients); chest pain (5 patients); and arthritis (17 patients).

The Lupus Activity Index showed the following positive symptoms of fibromyalgia patients: Fatigue (19 patients); joint pain (18 patients); mild rash (7 patients); mild neurological symptoms (5 patients); pleuritic chest pain (3 patients); Raynaud's phenomenon (3 patients); and shortness of breath (3 patients).

Employing the Systemic Lupus Activity Measure (SLAM), fibromyalgia patients complained of fatigue (20 patients); myalgias (20 patients); headaches (15 patients); joint pain (14 patients); cortical dysfunction (9 patients); abdominal pain (8 patients); oral ulcers (6 patients); rash (6 patients); shortness of breath (6 patients); alopecia (5 patients); Raynaud's (4 patients); and mild lymph node enlargement (1 patient).

### DISCUSSION

Patients with FMS can experience symptoms that are also associated with SLE and are specifically tested for on the SELENA-SLEDAI disease activity scale. When assessing for SLE, it is important to note that patients may also have undiagnosed fibromyalgia and therefore, should be assessed by experienced rheumatologists to rule out that pathology. The consequences of concomitant FMS with lupus have likely led to failed lupus drug trials that otherwise would have met their primary endpoint.

For example, Edratide (hCDR1) is a peptide derived from the complementarity determining region 1 (CDR1) of a monoclonal anti-DNA antibody that can bind to MHC class II molecules. (27) As a potential treatment for SLE, it works by modulating the immune response to increase the production of regulatory T cells, helping to relieve some of the autoimmune processes associated with SLE. In a phase II RCT, Urowitz et al. (28) evaluated the efficacy of Edratide in patients with mild-to-moderate SLE. They found the drug to be safe and well tolerated, yet it failed to meet primary endpoints based on SLEDAI-2K and Adjusted Mean SLEDAI (AMS). However, the secondary endpoint, which used the BILAG disease activity scale, was met for some of the subgroup dose levels. One potential reason for the failure to meet the primary endpoints could be because SLEDAI improvement can be influenced by subjects with FMS, as they frequently experience symptoms that are a part of the SLEDAI index.

### Conclusion

The coexistence of FMS and SLE presents a significant clinical challenge due to the overlap of symptoms between these two conditions. Patients with FMS often experience symptoms that are included in the SELENA-SLEDAI disease activity scale, which is used to assess lupus disease activity. Consequently, individuals with both FMS and SLE may frequently report these symptoms, and their presence can sometimes give the impression of resistance to treatment.

This overlap of symptoms has notable implications, particularly in the context of clinical drug trials for lupus. If patients with both SLE and FMS are included in such trials, the outcomes may appear less favorable, potentially leading to the misinterpretation that a new drug is ineffective. This is because the symptoms associated with FMS, such as “arthritis”-like pain, facial numbness, and cognitive changes, can be challenging to distinguish from lupus-related symptoms in these trials.

A study conducted by Wolfe et al. in 2014 highlighted this issue. It revealed that a significant portion of patients diagnosed with lupus by rheumatologists, approximately 31%, also met the criteria for FMS according to the 2010 American College of Rheumatology criteria for diagnosis. This underscores the prevalence of FMS among SLE patients and the potential for its symptoms to confound disease activity assessments.

In reality, new treatments for lupus may indeed be effective, but their efficacy might be masked when patients with concomitant FMS are included in the study. Therefore, it becomes essential for clinical trials and research studies on lupus to carefully consider the inclusion criteria and account for the presence of fibromyalgia. One such way to exclude patients with FMS is to use a one-page form from the American College of Rheumatology that patients can fill out. By identifying 12 or more points on the 31-point scale, they meet the clinical criteria for fibromyalgia.

Certain symptoms on the SELENA-SLEDAI scale, including headaches, paresthesia, etc., are common in fibromyalgia patients and may mask the effectiveness of lupus therapy and disease activity assessment. Excluding patients with concomitant fibromyalgia from lupus clinical trials can help provide a more accurate assessment of the drug's effectiveness in treating lupus-specific symptoms. Additionally, using alternative or supplementary measures to assess lupus disease activity that are less influenced by fibromyalgia-related symptoms may be necessary to obtain a more precise evaluation. The SELENA-SLEDAI is a helpful instrument if concomitant fibromyalgia is taken into account and excluded from the analysis.

### Conclusion

Symptoms of fibromyalgia can sometimes resemble those of other rheumatic diseases including lupus. Because many patients with lupus have concomitant fibromyalgia, it is difficult to separate the fibromyalgia component. Fibromyalgia symptoms can easily contaminate lupus activity indices including the SELENA-SLEDAI and the BILAG. Possible modifications of these indices should be evaluated in studies that include lupus patients and those with concomitant fibromyalgia to further refine the assessment tools.

Excluding patients with concomitant fibromyalgia might make the SELENA-SLEDAI and the BILAG more accurate assessment tools and exclude patients who are not as likely to respond to new lupus treatment.

Using the 2010 and 2016 American College of Rheumatology criteria for the diagnosis of fibromyalgia, including a simple one-page form for assessing the presence of the illness, concomitant fibromyalgia can be determined in lupus patients.

Unless fibromyalgia is excluded lupus treatment and its effectiveness will continue to be compromised by the relatively frequent presence of concomitant fibromyalgia. This makes the SELENA-SLEDAI and BILAG unreliable instruments for assessing the effectiveness of lupus interventions.

### REFERENCES

1. Hruskova Z, Tesar V. Lessons learned from the failure of several recent trials with biologic treatment in systemic lupus erythematosus. Expert opinion on biological therapy. 2018; 18 (9).
2. Mahieu M, Strand V, Simon L, Lipsky P, Ramsey-Goldman R. A critical review of clinical trials in systemic lupus erythematosus. Lupus. 2016; 25 (10).
3. Dolgin E. Lupus in crisis: as failures pile up, clinicians call for new tools. Nature biotechnology. 2019; 37 (1).
4. Wolfe F, Petri M, Alarcón G, Goldman J, Chakravarty E, Katz R, et al. Fibromyalgia, systemic lupus erythematosus (SLE), and evaluation of SLE activity. The Journal of rheumatology. 2009; 36 (1).
5. Middleton G, McFarlin J, Lipsky P. The prevalence and clinical impact of fibromyalgia in systemic lupus erythematosus. Arthritis and rheumatism. 1994; 37 (8).
6. Gladman D, Urowitz M, Gough J, Mackinnon A. Fibromyalgia is a major contributor to quality of life in lupus. The Journal of rheumatology. 1997; 24 (11).
7. Akkasilpa S, Goldman D, Magder L, Petri M. Number of fibromyalgia tender points is associated with health status in patients with systemic lupus erythematosus. The Journal of rheumatology. 2005; 32 (1).
8. Handa R, Aggarwal P, Wali J, Wig N, Dwivedi S. Fibromyalgia in Indian patients with SLE. Lupus. 1998; 7 (7).
9. Valencia-Flores M, Cardiel M, Santiago V, Resendiz M, Castaño V, Negrete O, et al. Prevalence and factors associated with fibromyalgia in Mexican patients with systemic lupus erythematosus. Lupus. 2004; 13 (1).
10. Pettersson S, Lövgren M, Eriksson L, Moberg C, Svenungsson E, Gunnarsson I, et al. An exploration of patient-reported symptoms in systemic lupus erythematosus and the relationship to health-related quality of life. Scandinavian journal of rheumatology. 2012; 41 (5).
11. Leuchten N, Milke B, Winkler-Rohlfing B, Daikh D, Dörner T, Johnson S, et al. Early symptoms of systemic lupus erythematosus (SLE) recalled by 339 SLE patients. Lupus. 2018; 27 (9).
12. Häuser W, Ablin J, Fitzcharles M, Littlejohn G, Luciano J, Usui C, et al. Fibromyalgia. Nature reviews. Disease primers. 2015; 1.
13. Bair M, Krebs E. Fibromyalgia. Annals of internal medicine. 2020; 172 (5).
14. Murphy C, Yee C, Gordon C, Isenberg D. From BILAG to BILAG-based combined lupus assessment-30 years on. Rheumatology (Oxford, England). 2016; 55 (8).
15. Isenberg D, Allen E, Farewell V, D'Cruz D, Alarcón G, Aranow C, et al. An assessment of disease flare in patients with systemic lupus erythematosus: a comparison of BILAG 2004 and the flare version of SELENA. Annals of the rheumatic diseases. 2011; 70 (1).
16. Lopez R, Davidson J, Beeby M, Egger P, Isenberg D. Lupus disease activity and the risk of subsequent organ damage and mortality in a large lupus cohort. Rheumatology (Oxford, England). 2012; 51 (3).
17. Bennett R, Jones J, Turk D, Russell I, Matallana L. An internet survey of 2,596 people with fibromyalgia. BMC musculoskeletal disorders. 2007; 8.
18. Siracusa R, Paola R, Cuzzocrea S, Impellizzeri D. Fibromyalgia: Pathogenesis, Mechanisms, Diagnosis and Treatment Options Update. International journal of molecular sciences. 2021; 22 (8).
19. Wolfe F, Clauw D, Fitzcharles M, Goldenberg D, Katz R, Mease P, et al. The American College of Rheumatology preliminary diagnostic criteria for fibromyalgia and measurement of symptom severity. Arthritis care & research. 2010; 62 (5).
20. Wolfe F, Clauw D, Fitzcharles M, Goldenberg D, Häuser W, Katz R, et al. 2016 Revisions to the 2010/2011 fibromyalgia diagnostic criteria. Seminars in arthritis and rheumatism. 2016; 46 (3).
21. Wolfe F, Ross K, Anderson J, Russell I, Hebert L. The prevalence and characteristics of fibromyalgia in the general population. Arthritis and rheumatism. 1995; 38 (1).
22. Mikdashi J, Nived O. Measuring disease activity in adults with systemic lupus erythematosus: the challenges of administrative burden and responsiveness to patient concerns in clinical research. Arthritis research & therapy. 2015; 17 (1).
23. Petri M, Kim M, Kalunian K, Grossman J, Hahn B, Sammaritano L, et al.

Combined oral contraceptives in women with systemic lupus erythematosus. The New England journal of medicine. 2005; 353 (24).
24. Gladman D, Ibañez D, Urowitz M. Systemic lupus erythematosus disease activity index 2000. The Journal of rheumatology. 2002; 29 (2).
25. Jump R, Robinson M, Armstrong A, Barnes E, Kilbourn K, Richards H. Fatigue in systemic lupus erythematosus: contributions of disease activity, pain, depression, and perceived social support. The Journal of rheumatology. 2005; 32 (9).
26. Katz R, Polyak Wokurka J, Small B. The SELENA SLEDAI Is an Imperfect Measure of Lupus Disease Activity in Patients with Concomitant Fibromyalgia [abstract]. Arthritis Rheumatol.; 2019.
27. 2023; Pageshttps://drugs.ncats.io/drug/38PLP07BKC.
28. Urowitz M, Isenberg D, Wallace D. Safety and efficacy of hCDR1 (Edratide) in patients with active systemic lupus erythematosus: results of phase II study. Lupus science & medicine. 2015; 2 (1).

### Example 2: Concomitant Fibromyalgia in Patients with Other Rheumatic Diseases and Response to Treatment

### Background/Purpose:

Fibromyalgia Syndrome (FMS) is a pathology that is under-diagnosed in the clinical population and can be present alongside other rheumatic diseases, including Systemic Lupus Erythematous (SLE), Sjogren's Syndrome, and rheumatoid factor negative Rheumatoid Arthritis. This phenomenon is known as concomitant fibromyalgia, and its presence can lead to difficulties in treating rheumatic diseases, including successful pharmaceutical trials.

### Methods:

There were four groups evaluated in this study. The first group contained FMS patients not diagnosed with another rheumatic disease. These patients were diagnosed by an experienced rheumatologist using the 2010 and 2016 American College of Rheumatology (ACR) diagnostic criteria. The second group consisted of patients diagnosed with rheumatic diseases other than FMS, such as SLE, rheumatoid arthritis, psoriatic arthritis, and ankylosing spondylitis, but had concomitant FMS based on the Polysymptomatic Distress Scale (PDS). (15) To obtain a concomitant FMS diagnosis, patients had to score at least 12 points on the 0-to-31-point PDS established by the 2010 and 2016 ACR diagnostic criteria for fibromyalgia. The calculation of this score is determined through the addition of the Widespread Pain Index (WPI) and the Symptom Severity (SS) scale. The third group was made up of patients diagnosed with a non-FMS rheumatic disease who scored a 10 or 11 on the PDS. These patients were classified as having intermediate concomitant FMS. Lastly, the fourth group consisted of patients diagnosed with a non-FMS rheumatic disease that did not have concomitant FMS (a score of less than 10 on the PDS).

The FMS and non-FMS syndrome rheumatic disease patient groups did not significantly differ in age or education. In the FMS group, there were 191 patients (160 females and 31 males). In the control non-fibromyalgia syndrome rheumatic disease group, there were 130 patients (82 females and 48 males). The mean age for the fibromyalgia patient was 52.45 years for females and 50.39 years for males. The mean ages in the controls were 55.23 years for females and 54.02 years for males.

The following information was collected and displayed in Table 1.

Descriptive statistics (N, mean, SD, median, min, max) for the responses to “How severe is the pain?” Descriptive statistics (N, mean, SD, median, min, max) for the responses to “How are you responding to treatment?” A Kruskall-Wallis test to compare all 4 groups' pain scores and treatment response scores. Upon confirming that there's indeed a difference between all 4 groups for both scores, comparisons were done, using the Mann-Whitney test and the Bonferroni correction.

### Results

There were 503 patients analyzed in this study; 75% were female and 25% were male. Of these, 108/503 (22%) had FMS diagnosis, 273/503 (54%) were determined to have other rheumatic diseases but no concomitant FMS (FMS score <10), 39/503 (8%) were determined to have other rheumatic diseases and potential FMS (FMS score 10 or 11), and 83/503 (17%) were determined to have other rheumatic diseases and concomitant FMS (FMS score >11).

The mean pain level for FMS patients with no other disease was 6.3±2.06. Patients who had another rheumatic disease with no diagnosis of FMS displayed a pain level of 4±2.62. Patients who were on the borderline for concomitant fibromyalgia diagnosis and were determined to be in the intermediate group had a pain level of 6.2±1.99. The pain level for concomitant fibromyalgia patients was the highest at 6.7±1.97.

For treatment response, FMS patients with no other rheumatic disease responded poorly with an average response of 6.4±2.06. The response of patients with a rheumatic disease other than FMS was the highest at 8.3±1.72. Response levels for intermediate and normal concomitant FMS were 7.8±2.28 and 7.1±1.57 respectively.

The Kruskall-Wallis test for both scores showed a significant difference between the 4 diagnosis groups for the pain score (p<0.0001) and the response to treatment score (p<0.0001), indicating that indeed there are differences in pain sensation and response to treatment between the 4 diagnosis groups. When multiple comparisons were performed, the ones that were statistically significantly different for the pain score were:
Concomitant FMS vs No Concomitant FMS (p<0.0001)
No Concomitant FMS vs FMS (p<0.0001)
No Concomitant FMS vs Intermediate/potential Concomitant FMS (p<0.0001)

TABLE 1
Data collected from questionnaires given to patients in each diagnosis group.

| | | | Rheumatic Disease | | Rheumatic Disease with | | Rheumatic Disease | |
|---|---|---|---|---|---|---|---|---|
| | | | with No Concomitant | | Intermediate Concomitant | | with Concomitant | |
| | FMS | | FMS (PDS < 10) | | FMS (PDS 10-11) | | FMS (PDS > 11) | |
| How severe is | | | | | | | | |
| the pain? | | | | | | | | |
| N | 100 | | 231 | | 32 | | 77 | |
| Mean | 6.3 | | 4 | | 6.2 | | 6.7 | |
| SD | 2.06 | | 2.62 | | 1.99 | | 1.97 | |
| Median | 6.5 | | 3 | | 6 | | 7 | |
| Min | 1 | | 1 | | 1 | | 2 | |
| Max | 10 | | 10 | | 9 | | 10 | |
| | n | % | n | % | n | % | n | % |
| 1 | 1 | 1 | 54 | 23.4 | 1 | 3.1 | 0 | 0 |
| 2 | 3 | 3 | 31 | 13.4 | 0 | 0 | 1 | 1.3 |
| 3 | 9 | 9 | 35 | 15.2 | 2 | 6.3 | 5 | 6.5 |
| 4 | 9 | 9 | 24 | 10.4 | 4 | 12.5 | 6 | 7.8 |
| 5 | 7 | 7 | 19 | 8.2 | 3 | 9.4 | 10 | 13 |
| 6 | 21 | 21 | 18 | 7.8 | 7 | 21.9 | 11 | 14.3 |
| 7 | 17 | 17 | 19 | 8.2 | 5 | 15.6 | 15 | 19.5 |
| 7.5 | 0 | 0 | 1 | 0.4 | 0 | 0 | 0 | 0 |
| 8 | 22 | 22 | 18 | 7.8 | 6 | 18.8 | 14 | 18.2 |
| 8.5 | 0 | 0 | 0 | 0 | 1 | 3.1 | 0 | 0 |
| 9 | 7 | 7 | 6 | 2.6 | 3 | 9.4 | 11 | 14.3 |
| 10 | 4 | 4 | 6 | 2.6 | 0 | 0 | 4 | 5.2 |
| How are you | | | | | | | | |
| responding | | | | | | | | |
| to treatment? | | | | | | | | |
| N | 21 | | 39 | | 5 | | 7 | |
| Mean | 6.4 | | 8.3 | | 7.8 | | 7.1 | |
| SD | 2.06 | | 1.72 | | 2.28 | | 1.57 | |
| Median | 7 | | 9 | | 8 | | 8 | |
| Min | 3 | | 3 | | 5 | | 5 | |
| Max | 10 | | 10 | | 10 | | 9 | |
| | n | % | n | % | n | % | n | % |
| 1 | 0 | 0 | 0 | 0 | 0 | 0 | 0 | 0 |
| 2 | 0 | 0 | 0 | 0 | 0 | 0 | 0 | 0 |
| 3 | 2 | 9.5 | 2 | 5.1 | 0 | 0 | 0 | 0 |
| 4 | 2 | 9.5 | 0 | 0 | 0 | 0 | 0 | 0 |
| 5 | 4 | 19 | 1 | 2.6 | 1 | 20 | 2 | 28.6 |
| 6 | 1 | 4.8 | 1 | 2.6 | 1 | 20 | 0 | 0 |
| 7 | 6 | 28.6 | 3 | 7.7 | 0 | 0 | 1 | 14.3 |
| 8 | 3 | 14.3 | 12 | 30.8 | 1 | 20 | 3 | 42.9 |
| 9 | 1 | 4.8 | 10 | 25.6 | 0 | 0 | 1 | 14.3 |
| 10 | 2 | 9.5 | 10 | 25.6 | 2 | 40 | 0 | 0 |

### DISCUSSION

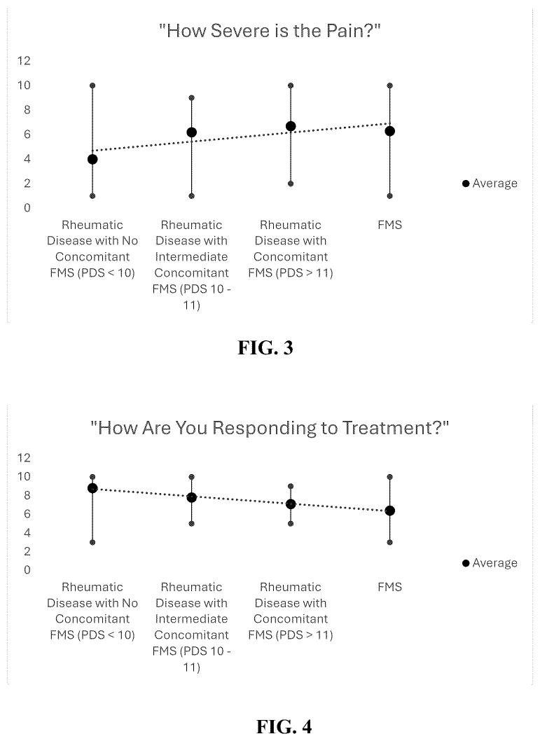

There was a clear difference in patients' reported pain levels and response to therapy in the rheumatic disease patients who had concomitant FMS and those who did not. Having concomitant FMS appears to be associated with higher pain scores and a worse response to treatment. See FIGS. 3 and 4. FIG. 3—Pain increases in severity as PDS scores increase.

### FIG. 4—Response to Treatment Decreases as PDS Scores Increase.

Because of the influence concomitant FMS has in rheumatic diseases, it is more difficult to treat the non-FMS pathology. There is not a clear picture of disease profile, so knowing the dosage and treatment regimen for the non-FMS pathology can be difficult to deduce. Not only does concomitant FMS make treatment plans more difficult to develop, but also can cause pharmaceutical trials to fail if that patient population is not excluded.

For example, symptoms of FMS can sometimes resemble those of SLE. Because many patients with SLE have concomitant fibromyalgia (8-12), it may be difficult to separate the fibromyalgia component. Fibromyalgia symptoms can easily contaminate lupus activity indices including the SELENA-SLEDAI and the BILAG. Excluding those patients with concomitant fibromyalgia would make the SELENA-SLEDAI and the BILAG more accurate assessment tools and exclude patients who are not as likely to respond to new lupus treatment. Using the 2010 and 2016 American College of Rheumatology criteria for the diagnosis of FMS, including a one-page form for assessing the presence of the illness, concomitant FMS can be determined in lupus patients. Unless FMS is excluded from pharmaceutical trials, lupus and other rheumatic disease drug treatments and effectiveness will continue to be compromised.

At a minimum, failure to take concomitant fibromyalgia into account is likely to result in measured outcome variables that are invalid for establishing the value of new therapeutic agents. Failure to make the distinction between SLE and SLE coupled with concomitant FMS can create measurement errors that covertly mask the real benefits of the clinical drug trial. An important task in future efficacy research for any rheumatology drug trials will be to screen out those with concomitant FMS before the clinical trial starts. This study has shed light on the clinical impact of concomitant FMS, a generally unmeasured contaminant in clinical trials, based on the efficacy of standard-of-care medication in a clinical setting.

### Conclusion

By identifying concomitant fibromyalgia using a one-page sheet for patients to fill out that has been validated by the 2016 American College of Rheumatology fibromyalgia diagnostic criteria, rheumatic disease patients can be evaluated more accurately as to whether they are likely to respond to treatment for the underlying rheumatic disease.

Without identifying concomitant fibromyalgia in those with other rheumatic diseases, treatment failure and side effects will be much more common.

### REFERENCES

1. Bennett R, Jones J, Turk D, Russell I, Matallana L. An internet survey of 2,596 people with fibromyalgia. BMC musculoskeletal disorders. 2007; 8.
2. Siracusa R, Paola R, Cuzzocrea S, Impellizzeri D. Fibromyalgia: Pathogenesis, Mechanisms, Diagnosis and Treatment Options Update. International journal of molecular sciences. 2021; 22 (8).
3. Wolfe F, Clauw D, Fitzcharles M, Goldenberg D, Katz R, Mease P, et al. The American College of Rheumatology preliminary diagnostic criteria for fibromyalgia and measurement of symptom severity. Arthritis care & research. 2010; 62 (5).
4. Wolfe F, Clauw D, Fitzcharles M, Goldenberg D, Häuser W, Katz R, et al. 2016 Revisions to the 2010/2011 fibromyalgia diagnostic criteria. Seminars in arthritis and rheumatism. 2016; 46 (3).
5. Wolfe F, Ross K, Anderson J, Russell I, Hebert L. The prevalence and characteristics of fibromyalgia in the general population. Arthritis and rheumatism. 1995; 38 (1).
6. Duffield S, Miller N, Zhao S, Goodson N. Concomitant fibromyalgia complicating chronic inflammatory arthritis: a systematic review and meta-analysis. Rheumatology (Oxford, England). 2018; 57 (8).
7. Mease P. Fibromyalgia, a missed comorbidity in spondyloarthritis: prevalence and impact on assessment and treatment. Current opinion in rheumatology. 2017; 29 (4).
8. Middleton G, McFarlin J, Lipsky P. The prevalence and clinical impact of fibromyalgia in systemic lupus erythematosus. Arthritis and rheumatism. 1994; 37 (8).
9. Gladman D, Urowitz M, Gough J, Mackinnon A. Fibromyalgia is a major contributor to quality of life in lupus. The Journal of rheumatology. 1997; 24 (11).
10. Akkasilpa S, Goldman D, Magder L, Petri M. Number of fibromyalgia tender points is associated with health status in patients with systemic lupus erythematosus. The Journal of rheumatology. 2005; 32 (1).
11. Handa R, Aggarwal P, Wali J, Wig N, Dwivedi S. Fibromyalgia in Indian patients with SLE. Lupus. 1998; 7 (7).
12. Valencia-Flores M, Cardiel M, Santiago V, Resendiz M, Castaño V, Negrete O, et al. Prevalence and factors associated with fibromyalgia in Mexican patients with systemic lupus erythematosus. Lupus. 2004; 13 (1).
13. Wolfe F, Petri M, Alarcón G, Goldman J, Chakravarty E, Katz R, et al. Fibromyalgia, systemic lupus erythematosus (SLE), and evaluation of SLE activity. The Journal of rheumatology. 2009; 36 (1).
14. Taylor J, Skan J, Erb N, Carruthers D, Bowman S, Gordon C, et al. Lupus patients with fatigue—is there a link with fibromyalgia syndrome? Rheumatology (Oxford, England). 2000; 39 (6).
15. Wolfe F, Walitt B, Rasker J, Katz R, Häuser W. The Use of Polysymptomatic Distress Categories in the Evaluation of Fibromyalgia (FM) and FM Severity. The Journal of rheumatology. 2015; 42 (8).

### Example 3: The Effect of Concomitant Fibromyalgia on BILAG Assessment of Lupus Activity

### In-Office Study of BILAG Questionnaire

In a recent study, 43 patients meeting the 2011 ACR criteria for the diagnosis of FMS took an in-office survey listing various symptoms that are part of the British Isles Activity Group (BILAG) lupus assessment. Symptoms experienced included arthralgias (40 patients); myalgias (40 patients); fatigue (34 patients); episodic migrainous headaches (33 patients); small mucosal ulcers (16 patients); dyspnea (18 patients); nausea (18 patients); intermittent chest pain (16 patients); swollen fingers (10 patients); rash (9 patients); paresthesia (9 patients); Raynaud's Phenomenon (7 patients); lymphadenopathy (7 patients); alopecia (5 patients); tendinitis (4 patients); and weight loss (2 patients). Patients sometimes endorsed other symptoms, but not commonly. The results are summarized in Table 2.

TABLE 2
Patients with FMS tend to
report symptoms common to SLE

| | # of FMS patients |
|---|---|
| | who answered |
| Common SLE Symptoms | yes (%) |
| Arthralgias | 40 (93) |
| Myalgias | 40 (93) |
| Fatigue | 34 (79) |
| Episodic Migrainous Headaches | 33 (77) |
| Dyspnea | 18 (42) |
| Nausea | 18 (42) |
| Small Mucosal Ulcers | 16 (37) |
| Intermittent Chest Pain | 16 (37) |
| Swollen Fingers | 10 (23) |
| Rash | 9 (21) |
| Paresthesias | 9 (21) |
| Raynaud's Phenomenon | 7 (16) |
| Lymphadenopathy | 7 (16) |
| Alopecia | 5 (12) |
| Tendinitis | 4 (9) |
| Weight Loss | 2 (5) |

This data shows that there are common symptoms between the two pathologies and fibromyalgia can be present in patients with SLE, despite being undiagnosed by rheumatologists. The most common symptoms between the two diseases are arthralgias, myalgias, fatigue, and headaches, all of which were reported by over 75% of participants.

### DISCUSSION

When assessing for SLE, it is important to note that patients may also have undiagnosed fibromyalgia and therefore, should be assessed by experienced rheumatologists to rule out that pathology. The consequences of concomitant FMS with lupus have likely led to failed lupus drug trials that otherwise would have met their primary endpoint.

For example, Abatacept is a medication that blocks T-cell activation by binding to CD80/86 on antigen-presenting cells and was studied as a potential treatment for non-renal SLE in a phase IIb RCT involving 175 patients with active musculoskeletal, cutaneous, or cardiovascular/respiratory SLE. (34) The trial's primary endpoint, which focused on the development of new SLE flares, was not met, with similar flare rates in the abatacept and placebo groups. However, post-hoc analyses showed that abatacept reduced severe flares in certain patients, especially those with polyarthritis. Additionally, abatacept led to significant improvements in quality of life, fatigue, and sleep quality. When studying the patient selection of this trial, over half of the subjects experienced polyarthritis as the primary manifestation. This symptom is common among patients with FMS, as was seen in an in-office study, and therefore, patients with this symptom in the abatacept trial may have had concomitant fibromyalgia that contaminated the results, leading to the failure. According to an analysis of the drug trial by Merrill et al., (34) the study aimed to include a more homogeneous SLE population with specific organ involvement criteria, but the strict definition of flares based on the BILAG scale made it challenging to achieve the primary efficacy endpoint. This is another reason why strict adherence to the BILAG scale, without consideration of concomitant fibromyalgia, can lead to failed lupus drug trials.

Another example of concomitant fibromyalgia potentially causing lupus drug trial failures can be seen with epratuzumab, a monoclonal antibody targeting CD22 on B-cells. (35) The EMBLEM study, a phase IIb trial, used the BILAG-Based Composite Lupus Assessment (BICLA) as the primary endpoint. BICLA measures improvement in baseline disease activity, no disease worsening, and no treatment failure to be considered a responder. Epratuzumab treatment in EMBLEM showed greater BICLA response at 12 weeks for all doses compared to placebo, but it was not statistically significant. One potential reason for the lack of statistical significance could be because BILAG improvement was the dependent variable used which, as argued above, can be influenced by concomitant fibromyalgia. Therefore, treatment with epratuzumab could have improved symptoms related to SLE, but since it had no effect, or possibly worsened the effect, on patients with concomitant FMS, it resulted in results that lacked statistical significance.

### Conclusion

The high failure rate of clinical drug trials for SLE over the past several decades has been a persistent challenge in the field of rheumatology. While the complexity of SLE and the difficulty in measuring disease activity have been widely acknowledged as contributing factors to these failures, the presence of concomitant fibromyalgia has been underappreciated as a potential confounder. Concomitant fibromyalgia plays an important role in masking the therapeutic benefits of medication, and it is anticipated that standard-of-care medication is associated with less favorable clinical outcomes when SLE is coupled with concomitant fibromyalgia.

This example has discussed the significant influence of concomitant FMS on the assessment of SLE disease activity, particularly when using established tools like the BILAG index. Patients with SLE and FMS often experience overlapping symptoms such as pain, fatigue, and cognitive dysfunction, which can lead to misinterpretations of disease activity and hinder the progress of lupus drug trials. A key finding highlighted is that a substantial portion of SLE patients, up to 45%, may have undiagnosed FMS, which can significantly affect the interpretation of clinical trial results. This is particularly concerning as many SLE drug trials rely on specific disease activity indices like BILAG, SELENA-SLEDAI, and SLAM, which may not adequately account for the presence of concomitant FMS.

The examples of Abatacept and Epratuzumab trials presented in this example illustrate how concomitant FMS may have contributed to the failure of lupus drug trials, as traditional endpoints like BILAG improvement may not accurately reflect the true impact of these treatments, especially when FMS symptoms are involved.

In light of these findings, researchers, clinicians, and pharmaceutical companies involved in SLE drug research must consider the potential presence of concomitant FMS in trial participants. Developing more sophisticated and specific assessment tools that can distinguish between SLE-related symptoms and FMS-related symptoms is essential to improve the accuracy of disease activity measurement. Moreover, clinical trial designs need to account for the heterogeneity of SLE patients, including those with concomitant FMS, to ensure that therapeutic agents are appropriately targeting the intended pathology. A better understanding of the interplay between SLE and FMS and its impact on clinical trial outcomes is essential for advancing the field and ultimately providing more effective treatments for patients with SLE.

### REFERENCES

1. Mahieu M, Strand V, Simon L, Lipsky P, Ramsey-Goldman R. A critical review of clinical trials in systemic lupus erythematosus. Lupus. 2016; 25 (10).
2. Dolgin E. Lupus in crisis: as failures pile up, clinicians call for new tools. Nature biotechnology. 2019; 37 (1).
3. Wolfe F, Petri M, Alarcón G, Goldman J, Chakravarty E, Katz R, et al. Fibromyalgia, systemic lupus erythematosus (SLE), and evaluation of SLE activity. The Journal of rheumatology. 2009; 36 (1).
4. Middleton G, McFarlin J, Lipsky P. The prevalence and clinical impact of fibromyalgia in systemic lupus erythematosus. Arthritis and rheumatism. 1994; 37 (8).
5. Gladman D, Urowitz M, Gough J, Mackinnon A. Fibromyalgia is a major contributor to quality of life in lupus. The Journal of rheumatology. 1997; 24 (11).
6. Akkasilpa S, Goldman D, Magder L, Petri M. Number of fibromyalgia tender points is associated with health status in patients with systemic lupus erythematosus. The Journal of rheumatology. 2005; 32 (1).
7. Handa R, Aggarwal P, Wali J, Wig N, Dwivedi S. Fibromyalgia in Indian patients with SLE. Lupus. 1998; 7 (7).
8. Valencia-Flores M, Cardiel M, Santiago V, Resendiz M, Castaño V, Negrete O, et al. Prevalence and factors associated with fibromyalgia in Mexican patients with systemic lupus erythematosus. Lupus. 2004; 13 (1).
9. Pettersson S, Lövgren M, Eriksson L, Moberg C, Svenungsson E, Gunnarsson I, et
al. An exploration of patient-reported symptoms in systemic lupus erythematosus and the relationship to health-related quality of life. Scandinavian journal of rheumatology. 2012; 41 (5).
10. Leuchten N, Milke B, Winkler-Rohlfing B, Daikh D, Dörner T, Johnson S, et al. Early symptoms of systemic lupus erythematosus (SLE) recalled by 339 SLE patients. Lupus. 2018; 27 (9).
11. Häuser W, Ablin J, Fitzcharles M, Littlejohn G, Luciano J, Usui C, et al. Fibromyalgia. Nature reviews. Disease primers. 2015; 1.
12. Bair M, Krebs E. Fibromyalgia. Annals of internal medicine. 2020; 172 (5).
13. Murphy C, Yee C, Gordon C, Isenberg D. From BILAG to BILAG-based combined lupus assessment-30 years on. Rheumatology (Oxford, England). 2016; 55 (8).
14. Isenberg D, Allen E, Farewell V, D'Cruz D, Alarcón G, Aranow C, et al. An assessment of disease flare in patients with systemic lupus erythematosus: a comparison of BILAG 2004 and the flare version of SELENA. Annals of the rheumatic diseases. 2011; 70 (1).
15. Lopez R, Davidson J, Beeby M, Egger P, Isenberg D. Lupus disease activity and the risk of subsequent organ damage and mortality in a large lupus cohort. Rheumatology (Oxford, England). 2012; 51 (3).
16. Bennett R, Jones J, Turk D, Russell I, Matallana L. An internet survey of 2,596 people with fibromyalgia. BMC musculoskeletal disorders. 2007; 8.
17. Siracusa R, Paola R, Cuzzocrea S, Impellizzeri D. Fibromyalgia: Pathogenesis, Mechanisms, Diagnosis and Treatment Options Update. International journal of molecular sciences. 2021; 22 (8).
18. Wolfe F, Clauw D, Fitzcharles M, Goldenberg D, Katz R, Mease P, et al. The American College of Rheumatology preliminary diagnostic criteria for fibromyalgia and measurement of symptom severity. Arthritis care & research. 2010; 62 (5).
19. Wolfe F, Clauw D, Fitzcharles M, Goldenberg D, Häuser W, Katz R, et al. 2016 Revisions to the 2010/2011 fibromyalgia diagnostic criteria. Seminars in arthritis and rheumatism. 2016; 46 (3).
20. Wolfe F, Ross K, Anderson J, Russell I, Hebert L. The prevalence and characteristics of fibromyalgia in the general population. Arthritis and rheumatism. 1995; 38 (1).
21. Liang M, Socher S, Larson M, Schur P. Reliability and validity of six systems for the clinical assessment of disease activity in systemic lupus erythematosus. Arthritis and rheumatism. 1989; 32 (9).
22. Bombardier C, Gladman D, Urowitz M, Caron D, Chang C. Derivation of the SLEDAI. A disease activity index for lupus patients. The Committee on Prognosis Studies in SLE. Arthritis and rheumatism. 1992; 35 (6).
23. Strand V, Gladman D, Isenberg D, Petri M, Smolen J, Tugwell P. Outcome measures to be used in clinical trials in systemic lupus erythematosus. The Journal of rheumatology. 1999; 26 (2).
24. Symmons D, Coppock J, Bacon P, Bresnihan B, Isenberg D, Maddison P, et al.
Development and assessment of a computerized index of clinical disease activity in systemic lupus erythematosus. Members of the British Isles Lupus Assessment Group (BILAG). The Quarterly journal of medicine. 1988; 69 (259).
25. Hay E, Bacon P, Gordon C, Isenberg D, Maddison P, Snaith M, et al. The BILAG index: a reliable and valid instrument for measuring clinical disease activity in systemic lupus erythematosus. The Quarterly journal of medicine. 1993; 86 (7).
26. Isenberg D, Gordon C. From BILAG to BLIPS—disease activity assessment in lupus past, present and future. Lupus. 2000; 9 (9).
27. Isenberg D, Rahman A, Allen E, Farewell V, Akil M, Bruce I, et al. BILAG 2004. Development and initial validation of an updated version of the British Isles Lupus Assessment Group's disease activity index for patients with systemic lupus erythematosus. Rheumatology (Oxford, England). 2005; 44 (7).
28. Isenberg D. Rituximab—it was the best of times, it was the worst of times. Autoimmunity reviews. 2012; 11 (11).
29. Hickman R, Hira-Kazal R, Yec C, Toescu V, Gordon C. The efficacy and safety of rituximab in a chart review study of 15 patients with systemic lupus erythematosus. Clinical rheumatology. 2015; 34 (2).
30. Turner-Stokes T, Lu T, Ehrenstein M, Giles I, Rahman A, Isenberg D. The efficacy of repeated treatment with B-cell depletion therapy in systemic lupus erythematosus: an evaluation. Rheumatology (Oxford, England). 2011; 50 (8).
31. Leandro M, Cambridge G, Edwards J, Ehrenstein M, Isenberg D. B-cell depletion in the treatment of patients with systemic lupus crythematosus: a longitudinal analysis of 24 patients. Rheumatology (Oxford, England). 2005; 44 (12).
32. Reddy V, Jayne D, Close D, Isenberg D. B-cell depletion in SLE: clinical and trial experience with rituximab and ocrelizumab and implications for study design. Arthritis research & therapy. 2013; 15 Suppl 1 (Suppl 1).
33. Isenberg D, Rahman A. Systemic lupus erythematosus in 2013. Taking a closer look at biologic therapy for SLE. Nature reviews. Rheumatology. 2014; 10 (2).
34. Merrill J, Burgos-Vargas R, Westhovens R, Chalmers A, D'Cruz D, Wallace D, ct al. The efficacy and safety of abatacept in patients with non-life-threatening manifestations of systemic lupus erythematosus: results of a twelve-month, multicenter, exploratory, phase IIb, randomized, double-blind, placebo-controlled trial. Arthritis and rheumatism. 2010; 62 (10).
35. Wallace D, Kalunian K, Petri M, Strand V, Houssiau F, Pike M, et al. Efficacy and safety of epratuzumab in patients with moderate/severe active systemic lupus erythematosus: results from EMBLEM, a phase IIb, randomised, double-blind, placebo-controlled, multicentre study. Annals of the rheumatic diseases. 2014; 73 (1).

### Example 4: Disease Activity Score (DAS) for Measuring Rheumatoid Arthritis (RA) Activity Skewed by Concomitant Fibromyalgia in RA Patients

The Disease Activity Score (DAS) is a widely used method for measuring rheumatoid arthritis (RA) activity, but could the results of DAS scoring be skewed by concomitant fibromyalgia in RA patients? Although fibromyalgia patients do not have joint swelling, they often have multiple tender joints (and muscles) and report a high disease activity score.

Methods: 16 fibromyalgia patients and 28 rheumatoid arthritis patients were evaluated using the DAS28. The fibromyalgia patients met the 2010 ACR criteria for the diagnosis, and rheumatoid arthritis patients met 2010 ACR/EULAR criteria for the diagnosis of RA The DAS28 was administered by a rheumatologist or a trained rheumatology nurse.

Results: As shown in FIG. 5, the DAS score in rheumatoid arthritis patients was 2.82 (0.49 to 6.93). In fibromyalgia patients, the DAS score mean was 3.45 and varied from 1.25 to 4.63. The DAS score was higher in fibromyalgia patients overall because of either increased joint tenderness and/or a higher disease activity score during the previous week.

Conclusion: DAS scores may be elevated in fibromyalgia patients and could lead to a contamination of DAS data in those patients who might have concomitant fibromyalgia and rheumatoid arthritis. DAS is a time-honored method of measuring RA activity and used in clinical trials, but may be an imperfect measurement in those who also have fibromyalgia.

### REFERENCES

Mahieu M A, Strand V, Simon L S, Lipsky P E, Ramsey-Goldman R. A critical review of clinical trials in systemic lupus erythematosus. Lupus. 2016; 25 (10): 1122-1140. doi: 10.1177/0961203316652492
Oude Voshaar, M. A. H., ten Klooster, P. M., Taal, E., Wolfe, F., Vonkeman, H., Glas, C. A. W. and van de Laar, M. A. F. J. (2014), Linking Physical Function Outcomes in Rheumatology: Performance of a Crosswalk for Converting Health Assessment Questionnaire Scores to Short Form 36 Physical Functioning Scale Scores. Arthritis Care & Research, 66:1754-1758. https://doi.org/10.1002/acr.22357
Chen, W., Zhang, X., & Huang, W. (2016). Role of neuroinflammation in neurodegenerative diseases (Review). Molecular Medicine Reports, 13, 3391-3396. https://doi.org/10.3892/mmr.2016.4948
Wolfe F, Petri M, Alarcón G S, Goldman J, Chakravarty E F, Katz R S, Karlson E W. Fibromyalgia, systemic lupus erythematosus (SLE), and evaluation of SLE activity. J Rheumatol. 2009 January; 36 (1): 82-8. doi: 10.3899/jrheum.080212. PMID: 19004039; PMCID: PMC2944223.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of diagnosing lupus and fibromyalgia, comprising the steps:
a. Diagnosing a patient with lupus;
b. Testing the lupus patient for fibromyalgia;
c. Excluding the lupus patients who test positive for fibromyalgia in a clinical study; and
d. Treating the lupus patients without fibromyalgia with a lupus therapy in the clinical study.

2. The method of claim 1, wherein testing the lupus patient for fibromyalgia uses American College of Rheumatology (ACR) criteria.

3. The method of claim 1, wherein diagnosing the patient with lupus is by SELENA-SLEDAI for measuring the activity of systemic lupus.

4. The method of claim 3, wherein the treating the lupus patients without fibromyalgia with a lupus therapy is by BILAG (British Isles Lupus Assessment Group).

5. The method of claim 4, further comprising modifying indices in SELENA-SLEDAI and BILAG; and further refining assessment tools in response to a treatment for lupus.

6. The method of claim 5, wherein further comprising using the ACR diagnostic criteria scale and the lupus patients who scored 12 or more points on the ACR diagnostic criteria scale are diagnosed with having fibromyalgia.

7. The method of claim 6, further comprising using the ACR diagnostic criteria scale include lupus patients who scored 10 to 12 on that scale are intermediate in the determination of a fibromyalgia diagnosis.

8. The method of claim 7, further comprising evaluating patients with inflammatory rheumatic diseases in addition to fibromyalgia for treatment response based on a visual analog scale.
